(19) United States
(12) Patent Application Publication
(10) Pub. No.: US 2025/0073390 A1
(43) Pub. Date: Mar. 6, 2025

(54) DRUG DELIVERY DEVICE WITH DETACHMENT CAPABILITIES

(71) Applicant: Biograil APS, Roskilde (DK)

(72) Inventors: Nikolaj Skak, Roskilde (DK); Karsten Lindhardt, Roskilde (DK); Kamille Majken Dumong Erichsen, Roskilde (DK)

(21) Appl. No.: 18/728,044

(22) PCT Filed: Jan. 24, 2023
(86) PCT No.: PCT/EP2023/051722

§ 371 Date: Jul. 10, 2024

(30) Foreign Application Priority Data
Jan. 28, 2022 (DK) PA202270036

(51) Int. Cl. A61M 5/20; A61M 31/00

(52) U.S. Cl. CPC A61M 5/20; A61M 31/002

(57) ABSTRACT

A drug delivery device comprising a first body part, a second body part, a first attachment part connected to the first body part and having a first distal end and a second attachment part connected to the second body part and having a second distal end. and an actuator mechanism configured to rotate one of the first body part and/or the second body part about a central axis. The drug delivery device is configured to transition from an initial configuration to an attachment configuration, and from the attachment configuration to a release configuration.

The present disclosure relates to a drug delivery device and in particular to a drug delivery device for oral administration. The drug delivery device is advantageously configured for delivery of an active drug substance in the gastrointestinal tract including the stomach and/or intestines, such as the small intestines and/or the large intestines (colon).

### BACKGROUND

A number of low permeable and/or low water soluble active drug substances are currently delivered by i.e. subcutaneous, intradermal, intramuscular, rectal, vaginal or intravenous route. Oral administration has the potential for the widest patient acceptance and thus attempts to deliver low permeable and/or low water soluble active drug substances through the preferred oral route of administration has been tried but with limited success in particular due to lack of stability and limited absorption from the gastrointestinal tract.

Stability both relates to the stability of the active drug substance during manufacturing and storage of the delivery device, and to the stability of the active drug substance during the passage in the gastrointestinal tract before it become available for absorption.

Limited gastrointestinal absorption is due to the gastrointestinal wall barrier preventing active drug substance from being absorbed after oral dosing because of the low permeability of the active drug substance, which is for example due to pre-systemic metabolism, size and/or the charges and/or because of the water solubility of the active drug substance.

Multiple approaches to solve these stability and absorption challenges have been suggested, but an effective solution to the challenges remain unresolved.

### SUMMARY

Thus, there is an unmet need to provide a drug delivery device, which is capable of delivering drug substances for absorption in the gastrointestinal tissue. More generally, there remains a need for drug products and methods that enable enhanced drug delivery, when drug products are administered orally to patients.

Disclosed herein is a drug delivery device. The drug delivery device comprises a first body part and a second body part. In one or more example drug delivery devices, the drug delivery device comprises a first attachment part. The first attachment part can be connected to the first body part. The first attachment part can have a first distal end. In one or more example drug delivery devices, the drug delivery device comprises a second attachment part. The second attachment part can be connected to the second body part. The second attachment part can have a second distal end. In one or more example drug delivery devices, the drug delivery device comprises an actuator mechanism. The actuator mechanism is configured to rotate one or both of the first body part and/or the second body part about a central axis. In one or more example drug delivery devices, the drug delivery device is configured to transition from an initial configuration to an attachment configuration. In one or more example drug delivery devices, the drug delivery device is configured to transition from the attachment configuration to a release configuration. The transition from the initial configuration to the attachment configuration optionally comprises translating the first attachment part in a first direction about a first axis in relation to the first body part. The transition from the initial configuration to the attachment configuration optionally comprises rotating the first body part in relation to the second body part in a first actuating direction about the central axis. The transition from the attachment configuration to the release configuration optionally comprises one or more of translating the first attachment part in the first direction, and rotating the first body part in relation to the second body part in a first detachment direction opposite the first actuating direction.

It is an advantage of a drug delivery device that the drug delivery device or at least parts thereof, such as the first body part and the second body part, can be released from the patient's system efficiently, such as after delivering an active drug substance to the patient. This can reduce and/or eliminate any bodily response to the drug delivery device.

Further, the present disclosure may reduce negative impact on the gastrointestinal tissue at the site of engagement and/or during passage of the drug delivery device in the gastrointestinal tract.

The present disclosure allows for efficient releasing of drug delivery devices from a patient, such as after providing an active drug substance. In particular, the drug delivery device may be used for attachment of the drug delivery device into a patient's system. The drug delivery device can then be released from the patient via one or more of the methods and/or components discussed herein.

### BRIEF DESCRIPTION OF THE DRAWINGS

The above and other features and advantages of the present invention will become readily apparent to those skilled in the art by the following detailed description of example embodiments thereof with reference to the attached drawings, in which:

FIG. 1 shows a perspective view of an example drug delivery device,

FIG. 2 shows an exploded view of an example drug delivery device,

FIGS. 3A-3B show a perspective view of an example drug delivery device in an attachment configuration and a release configuration, and

FIGS. 4A-4B show a perspective view of an example drug delivery device in an attachment configuration and a release configuration.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments and the functionalities associated therewith. They are not intended as an exhaustive description of the invention or as a limitation on the scope of the invention or the physical appearance of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

Disclosed herein is a drug delivery device. The drug delivery device comprises a first body part and a second body part. In one or more example drug delivery devices, the drug delivery device comprises a first attachment part. The attachment part can be connected to the first body part. The attachment part can have a first distal end. In one or more example drug delivery devices, the drug delivery device comprises a second attachment part. The second attachment part can be connected to the second body part. The second attachment part can have a second distal end. In one or more example drug delivery devices, the drug delivery device comprises an actuator mechanism. The actuator mechanism is configured to rotate one of the first body part and/or the second body part about a central axis. In one or more example drug delivery devices, the drug delivery device is configured to transition from an initial configuration to an attachment configuration. In one or more example drug delivery devices, the drug delivery device is configured to transition from the attachment configuration to a release configuration. The transition from the initial configuration to the attachment configuration comprises translating the first attachment part in a first direction about a first axis in relation to the first body part. The transition from the initial configuration to the attachment configuration comprises rotating the first body part in relation to the second body part in a first actuating direction about the central axis. The transition from the attachment configuration to the release configuration can include one or more of translating the first attachment part in the first direction, and rotating the first body part in relation to the second body part in a first detachment direction opposite the first actuating direction.

In one or more example drug delivery devices, a drug delivery device comprises a first body part; a second body part; a first attachment part connected to the first body part and having a first distal end and a second attachment part connected to the second body part and having a second distal end; and an actuator mechanism configured to rotate one of the first body part and/or the second body part about a central axis; wherein the drug delivery device is configured to transition from an initial configuration to an attachment configuration, and from the attachment configuration to a release configuration; wherein the transition from the initial configuration to the attachment configuration comprises translating the first attachment part in a first direction about a first axis in relation to the first body part, and rotating the first body part in relation to the second body part in a first actuating direction about the central axis; and wherein the transition from the attachment configuration to the release configuration comprises one or more of translating the first attachment part in the first direction, and rotating the first body part in relation to the second body part in a first detachment direction opposite the first actuating direction.

In one or more example drug delivery devices, the transition from the attachment configuration to the release configuration can include rotating the first body part in relation to the second body part in the first actuating direction, such as for unidirectional release.

The drug delivery device may have a size and geometry designed to fit into a pharmaceutical composition for oral administration.

The drug delivery device may be configured to be taken into the body via the oral orifice. For example, the drug delivery device may be configured to be taken into the body in an initial configuration. Thus, the outer dimensions of the drug delivery device may be small enough for a user to swallow. The drug delivery device may be adapted to carry a drug substance, e.g., an active drug substance, into the body of the user, via the digestive system, so that the drug delivery device may e.g., travel from the mouth of the user into the stomach, via the oesophagus. The drug delivery device may be configured to further travel into the intestines from the stomach, and may optionally travel into the bowels and out through the rectum.

The drug delivery device may be configured to deliver the drug in any part of the digestive system of the user, where in one example it may be configured to deliver a drug substance into the stomach of the user. In another example, the drug delivery device may be adapted to initiate the drug delivery when the device has passed the stomach and has entered the intestine of the user. In other words, the drug delivery device may be configured to attach to a wall of the stomach or a wall of the intestines, e.g., depending on the desired release position of the active drug substance. For example, the drug delivery device may be configured to transition from the initial configuration to an attachment configuration to attach to the patient.

The attachment part(s) of the drug delivery device may be configured to interact with the inner surface linings of the gastrointestinal tract, so that the drug delivery device may e.g., be attached to the inner surface (mucous membrane) of the stomach, or alternatively to the mucous membrane of the intestines, such as in the attachment configuration. The attachment part(s), e.g. first attachment part and/or second attachment part, may be configured to interact with the mucous membranes, e.g., in order to fix or attach the drug delivery device, e.g., for a period of time, inside the body of the user. By attaching the drug delivery device, it will allow a drug substance to be delivered into a part of the digestive system, in order to provide a drug substance to the body of the user. The attachment part(s) may be configured to interact with the mucous membranes, e.g., in order to inject drug substance into the gastrointestinal tract wall.

In one or more example drug delivery devices, the drug delivery device may be configured with detachment capabilities. For example, the drug delivery device may be constructed in a way that releases the drug delivery device or at least parts thereof from internal tissues and/or internal surfaces, such as after distribution of the active drug substance, such as by transitioning from the attachment configuration to the release configuration.

For example, the drug delivery device may have an initial configuration. The initial configuration may be an initial position and/or an initial state, such as when the drug delivery device is taken into a patient, such as via swallowing. The initial configuration can be a configuration before any actuation of an actuator mechanism of the drug delivery device.

The drug delivery device may have an attachment configuration. The attachment configuration may be an attachment position and/or an attachment state. The attachment configuration may allow for the drug delivery device to attach to a surface in the patient, such as tissue, which can allow for delivery of the active drug substance. The attachment configuration can be a configuration for attachment of the drug delivery device to the patient. The attachment configuration may be different from the initial configuration. In the attachment configuration, the drug delivery device may remain attached to the patient. The attachment configuration can be a configuration after an actuation of the actuator mechanism of the drug delivery device.

The drug delivery device may include a release configuration. The release configuration may be a release position and/or a release state. The release configuration may allow for the drug delivery device or at least parts thereof, such as the first body part and the second body part, to release, such as detach, from a surface of the patient, such as tissue. The release configuration can be a configuration for releasing the drug delivery device from the patient. The release configuration can be a configuration after release of an active drug substance from the drug delivery device. The release configuration can allow for the drug delivery device to detach from, such as release from, such as loosen from, the patient, such as a structure of the patient and/or tissue of the patent. The release configuration can be different from the attachment configuration. The release configuration can be different from the initial configuration. The release configuration can be the same as the initial configuration, albeit later in time. The release configuration can occur after the attachment configuration.

In one or more example drug delivery systems, the drug delivery device may be configured to transition from the initial configuration to the attachment configuration. In one or more example drug delivery systems, the drug delivery device may be configured to transition from the attachment configuration to the release configuration.

The drug delivery device includes a first body part. The first body part may be a one-part or single-part body part, e.g., unitary. The first body part may be a two-part body part, i.e. the first body part may comprise a first primary body part and a first secondary body part. The first body part has an outer surface. A first recess may be formed in the outer surface of the first body part, such as discussed below.

In one or more example drug delivery devices, the first body part may define a first recess, such as a cavity, gap, slot, hole, aperture, retainer, first body recess, extending to an outer surface of the first body part. The first recess may be formed by solid walls on all sides except an outermost surface, which is open. For example, the first recess may be formed by a first wall, a second wall, and a bottom. The first wall may be opposite the second wall. The bottom may connect the first wall and the second wall. In one or more example drug delivery devices, the first wall and/or the bottom wall and/or the second wall are flat. In one or more example drug delivery devices, the first wall and/or the bottom wall and/or the second wall are curved. The open outermost surface may be curved to follow along with an outer surface of the drug delivery device.

The drug delivery device includes a second body part. The second body part may be a one-part or single-part body part, e.g., unitary. The second body part may be a two-part body part, i.e. the second body part may comprise a second primary body part and a second secondary body part. The second body part has an outer surface. A second recess may be formed in the outer surface of the second body part.

In one or more example drug delivery devices, the second body part may define a second recess (e.g., cavity, gap, slot, hole, aperture, retainer, first body recess) extending to an outer surface of the second body part. The second recess may be formed by solid walls on all sides except an outermost surface, which is open. For example, the second recess may be formed by a first wall, a second wall, and a bottom. The first wall may be opposite the second wall. The bottom may connect the first wall and the second wall. In one or more example drug delivery devices, the first wall and/or the bottom wall and/or the second wall are flat. In one or more example drug delivery devices, the first wall and/or the bottom wall and/or the second wall are curved. The open outermost surface may be curved to follow along with an outer surface of the drug delivery device.

In one or more example drug delivery devices, the second body part may not contain a second recess. For example, the second attachment part, such as the second spike component, may be directly attached to an outer surface of the second body part.

The first body part and the second body part may be rotatably connected. For example, the first body part and/or the second body part may be configured to rotate about a central axis. The central axis may extend longitudinally through the drug delivery device.

The first body part can be configured to rotate in a first actuating direction about the central axis with respect to the second body part. The second body part can be configured to rotate in a second actuating direction about the central axis with respect to the first body part. The first actuating direction may be opposite of the second actuating direction.

The first body part can be configured to rotate in a first detachment direction about the central axis with respect to the second body part. The second body part can be configured to rotate in a second detachment direction about the central axis with respect to the first body part. The first detachment direction may be opposite of the first actuating direction. The second detachment direction may be opposite of the second actuating direction. The first detachment direction may be opposite of the second detachment direction.

The drug delivery part may include one or more attachment part(s), e.g., a first attachment part and/or a second attachment, of the drug delivery device, the one or more attachment part(s) may be seen as any kind of attachment parts that may be capable of attaching the drug delivery device to a biological tissue, such as a stomach wall, a wall of the bowels and/or intestines of a human or animal body. The attachment part(s), e.g., first attachment part and/or second attachment, may be configured to attach the drug delivery device to a patient. The attachment part(s), e.g., first attachment part and/or a second attachment, may be configured to deliver an active drug substance.

The drug delivery device includes a first attachment part. The first attachment part may comprise a first base part and/or a first spike component, e.g. spike component, needle, spike, spear. The first attachment part has a first proximal end, e.g. proximal end, and a first distal end, e.g. distal end. The first attachment part has a first distal end. The attachment part can have a first proximal end. A first tip of the first spike component forms the first distal end. In other words, the first distal end is a first tip of the first spike component. The first base may be arranged at or constitute the first proximal end of the first attachment part.

The first spike component may have a length in the range from 1 mm to 15 mm such, as in the range from 3 mm to 10 mm. Thereby sufficient penetration into the internal tissue may be provided for while at the same time reducing the risk of damaging the internal tissue. The first distal end of the first attachment part may be provided with a tip configured to penetrate a biological tissue. The first distal end of the first attachment part may be provided with a gripping part configured to grip a biological tissue.

The first spike component may be straight and/or curved. The first spike component may include two or more straight portions formed at an angle. For example, the first spike component may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion.

The first attachment part may translatable attached to the first body part. The first attachment part may be configured to move with respect to the first body part. For example, the first attachment part may be rotationally attached to the first body part. The first attachment part may be rotationally connected within the first recess. In particular, the first attachment part may be rotationally attached within the first recess, the first recess being discussed in detail below.

In one or more example drug delivery devices, the first attachment part may include one or more components, e.g. parts, intermediates, pieces. One or more of the one or more components may be releasably connected to the drug delivery device, such as the first body part.

The first attachment part can be held within the first recess of the first body part. In one or more example drug delivery devices, the first attachment part can be translatable connected to the first body part. The first attachment part can be rotatably connected to the first body part. In an initial configuration, the first attachment part can be held fully within the first recess. This can be the initial position. In an attachment configuration and/or a release configuration, the first attachment part can may at least partially extend out of the first recess, which may, respectively, be the first attachment position and the first release position. Thus, a distal end of the first attachment part can translate, such as rotate, from within the first recess, such as in the first initial position, to outside of the first recess, such as the first attachment position and/or the first release position. This movement can occur during rotation of the first body part and/or may be a result of rotation of the first body part.

While rotational motion is discussed herein, it will be understood that other translations and/or motions can be used as well, e.g., as an alternative or in combination with rotational motion. For example, straight, longitudinal, radial, and circumferential motion can all be used to translate the connector component between the first position and the second position. Further, as used herein translate can include one or more of shifting, rotation, moving, and releasing.

In one or more example drug delivery devices, a proximal end of the first attachment part may include a first attachment point (e.g., attachment surface, attachment mechanism, attachment plane, attachment section, attachment component, attachment structure, attachment, connection, rotatable connection) to the first body part. For example, the first attachment part may include a first attachment point with the first body part. The first attachment point may be, for example a hinge. The first attachment point may be a pin. The type of attachment point is not limiting.

In one or more example drug delivery devices, the first attachment part can be configured to translate at the first attachment point with respect to the first body part. In one or more example drug delivery devices, the first attachment part can be configured to rotate at the first attachment point with respect to the first body part. The rotation of the first attachment part can be about a first axis in a first direction about the first axis and/or a second direction about the first axis. The second direction may be opposite of the first direction. For example, the first attachment part may be rotationally connected within the first recess along a first axis.

The first axis may be different from the central axis. The first axis may be perpendicular, such as orthogonal, from the central axis. The first axis may be angled from the central axis, e.g. at a first angle less than 30 degrees, such as from 0 to 20 degrees, or larger than 30 degrees, such as in the range from 45 degrees to 135 degrees. The first axis may be parallel with the central axis.

In one or more example drug delivery devices, the first attachment part may rotate on the first axis in order for the first attachment part to move perpendicular to the central axis. In certain embodiments, the first attachment part may rotate at an angle between perpendicular and parallel with respect to the central axis.

The first axis may be spaced away from the central axis, such as radially spaced away. For example, the first axis may be located on an outer surface of the drug delivery device. The first axis may be located within the first recess. The central axis may be located at a longitudinal center of the drug delivery device. In one or more example drug delivery devices, where the first axis is parallel with the central axis, the distance between the first axis and the central axis may be at least 1 mm.

Alternatively, or in conjunction, the first attachment part may translate in a non-rotational manner about the first axis in relation to the first body part.

The first attachment part, such as the first spike component, may have one or more openings providing access to a chamber in the first body part and/or the first attachment part. In one or more example drug delivery devices, the chamber is formed as a through-going bore in the first spike component. The chamber can be configured to hold, such as retain, an active drug substance.

In one or more example drug delivery devices, the chamber may be open from an inner volume of the drug delivery device and towards an outer part of the drug delivery device. In one or more examples, the chamber may be inside the first spike component so that when the first distal end of the first attachment part has penetrated the biological tissue, the active drug substance may be released from the chamber and into the biological tissue via the first spike component. This may e.g., be where the first spike component is a tubular part, which has a first distal end in fluid communication with the chamber.

The drug delivery device includes a second attachment part. The second attachment part may comprise a second base part and/or a second spike component, e.g. spike component, needle, spike, spear. The second attachment part has a second proximal end, e.g. proximal end, and a second distal end, e.g. distal end. The second attachment part has a second distal end. The second attachment part can have a second proximal end. A second tip of the second spike component forms the second distal end. In other words, the second distal end is a second tip of the second spike component. The second base may be arranged at or constitute the second proximal end of the second attachment part.

The second spike component may have a length in the range from 1 mm to 15 mm such, as in the range from 3 mm to 10 mm. Thereby sufficient penetration into the internal tissue may be provided for while at the same time reducing the risk of damaging the internal tissue. The second distal end of the second attachment part may be provided with a tip configured to penetrate a biological tissue. The second distal end of the second attachment part may be provided with a gripping part configured to grip a biological tissue.

The second spike component may be straight and/or curved. The second spike component may include two or more straight portions formed at an angle. For example, the second spike component may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion.

The second attachment part may translatable attached to the second body part. The first attachment part may be configured to move with respect to the second body part. For example, the second attachment part may be rotationally attached to the second body part. The second attachment part may be rotationally connected within the second recess. In particular, the second attachment part may be rotationally attached within the second recess.

In one or more example drug delivery devices, the second attachment part may include one or more components, e.g. parts, intermediates, pieces. One or more of the one or more components may be releasably connected to the drug delivery device, such as the second body part.

The second attachment part can be held within the second recess of the first body part. In one or more example drug delivery devices, the second attachment part can be translatable connected to the second body part. The second attachment part can be rotatably connected to the second body part. In an initial configuration, the second attachment part can be held fully within the second recess. This can be the initial position. In an attachment configuration and/or a release configuration, the second attachment part can may at least partially extend out of the second recess, which may, respectively, be the second attachment position and the second release position. Thus, a distal end of the second attachment part can translate, such as rotate, from within the second recess, such as in the second initial position, to outside of the second recess, such as the second attachment position and/or the second release position. This movement can occur during rotation of the second body part and/or may be a result of rotation of the second body part.

While rotational motion is discussed herein, it will be understood that other translations and/or motions can be used as well. For example, straight, longitudinal, radial, and circumferential motion can all be used to translate the connector component between the first position and the second position. Further, as used herein translate can include one or more of shifting, rotation, moving, and releasing.

In one or more example drug delivery devices, a proximal end of the second attachment part may include a second attachment point (e.g., attachment surface, attachment mechanism, attachment plane, attachment section, attachment component, attachment structure, attachment, connection, rotatable connection) to the second body part. For example, the second attachment part may include a second attachment point with the second body part. The second attachment point may be, for example a hinge. The second attachment point may be a pin. The type of attachment point is not limiting.

In one or more example drug delivery devices, the second attachment part can be configured to translate at the second attachment point with respect to the second body part. In one or more example drug delivery devices, the second attachment part can be configured to rotate at the second attachment point with respect to the second body part. The rotation of the second attachment part can be about a second axis in a first direction about the second axis and/or a second direction about the second axis. The second direction may be opposite of the first direction. For example, the second attachment part may be rotationally connected within the second recess along a first axis. The first direction about the second axis may be opposite of the first direction about the first axis.

The second axis may be different from the central axis. The second axis may be perpendicular, such as orthogonal, from the central axis. The second axis may be angled from the central axis, e.g. at a second angle less than 30 degrees, such as from 0 to 20 degrees, or larger than 30 degrees, such as in the range from 45 degrees to 135 degrees. The second axis may be parallel with the central axis. The second axis may be perpendicular, such as orthogonal, from the first axis. The second axis may be angled from the first axis, e.g. at a second angle less than 30 degrees, such as from 0 to 20 degrees, or larger than 30 degrees, such as in the range from 45 degrees to 135 degrees. The second axis may be parallel with the first axis.

In one or more example drug delivery devices, the second attachment part may rotate on the second axis in order for the second attachment part to move perpendicular to the central axis. In certain embodiments, the second attachment part may rotate at an angle between perpendicular and parallel with respect to the central axis.

The second axis may be spaced away from the central axis, such as radially spaced away. For example, the second axis may be located on an outer surface of the drug delivery device. The second axis may be located within the second recess. The central axis may be located at a longitudinal center of the drug delivery device. In one or more example drug delivery devices, where the second axis is parallel with the central axis, the distance between the second axis and the central axis may be at least 1 mm. In one or more example drug delivery devices, where the second axis is parallel with the first axis, the distance between the second axis and the second axis may be at least 1 mm. The first axis may coincide with the second axis.

Alternatively, or in conjunction, the second attachment part may translate in a non-rotational manner about the second axis in relation to the second body part.

The second attachment part, such as the second spike component, may have one or more openings providing access to a chamber in the second body part and/or the second attachment part. In one or more example drug delivery devices, the chamber is formed as a through-going bore in the second spike component. The chamber can be configured to hold, such as retain, an active drug substance.

In one or more example drug delivery devices, the chamber may be open from an inner volume of the drug delivery device and towards an outer part of the drug delivery device. In one or more examples, the chamber may be inside the second spike component so that when the second distal end of the second attachment part has penetrated the biological tissue, the active drug substance may be released from the chamber and into the biological tissue via the second spike component. This may e.g., be where the second spike component is a tubular part, which has a first distal end in fluid communication with the chamber.

In one or more example drug delivery devices, further, e.g., multiple, attachment parts may be utilized. They may be rotatably attached to the first body part and/or the second body part. The multiple further attachment parts may include the same components as discussed above with respect to the first attachment part and/or the second attachment part.

In one or more example drug delivery devices, the drug delivery device can include an actuator mechanism. The actuator mechanism is configured to rotate the first body part in relation to the second body part about the central axis of the drug delivery device. The actuator mechanism is configured to rotate the first body part in relation to the second body part about the central axis of the drug delivery device in the first actuating direction. The actuator mechanism may be, or may include a resilient part. The actuator mechanism may be a spring. The actuator mechanism can be configured to rotate one of the first body part and/or the second body part about the central axis.

In one or more example drug delivery devices, a first part of the actuator mechanism, such as the resilient part, may be connected to the first body part and a second part of the actuator mechanism, such as the resilient part, may be connected to the second body part. This means that the actuator mechanism, such as the resilient part, may be utilized to store energy, such as rotational energy or rotational force which is applied to the first body part and the second body part, where the energy is stored in the actuator mechanism, such as the resilient part. Furthermore, when the energy is released, the force may be released to both the first body part and the second body part, which in turn transfers the force to the first attachment part and the second attachment part. The actuator mechanism, such as the resilient part, may e.g., be in the form of a helical spiral spring (mainspring) and/or a spiral torsion spring, where the first body part may be wound relative to the second body part by rotating the first body part relative to the second body part. This stores energy in the mainspring by twisting the spiral tighter. The stored force of the mainspring may then rotate the first body part in the opposing direction as the mainspring unwinds, e.g. in response to a release of the first body part in relation to the second body part. Thus, the force of the mainspring may cause the first attachment part and the second attachment parts to travel in opposing directions, and where the attachment parts may pinch the biological tissue and either pinch the tissue or penetrate the tissue in order to attach the drug delivery device to the biological tissue.

For example, the actuator mechanism can be configured to transition the drug delivery device from the initial configuration to the attachment configuration. The actuator mechanism can be configured to translate the first attachment part and/or the second attachment part from the respective initial position the respective attachment position.

In one or more example drug delivery devices, the first body part is configured to rotate in a first actuating direction and/or the second body part is configured to rotate in a second actuating direction opposite to the first actuating direction. In other words, the first body part is configured to rotate in relation to the second body part in a first actuating direction about the central axis.

In one or more example drug delivery devices, release of the actuator mechanism can be configured to transition the drug delivery device from the attachment configuration to the release configuration. For example, the rotational force can be released or changed, which may cause the drug delivery device to transition from the attachment continuation to the release configuration.

The rotational connection between the first body part and the second body part allows the first body part to rotate relative to the second body part, such as in the first actuating direction and/or the first detachment direction, without the two parts separating from each other before the attachment part(s) interact with the internal tissue, such as mucous membranes. Such a connection may be obtained in a plurality of ways, where in one example the first body part has a plug connection and the second body part has a socket connection, where this plug and socket configuration allows the first body part to rotate relative to the second body part. A second example could be to provide a central shaft, such as an axle, that may be coaxial with the central axis, where the first body part and the second body part are configured to receive the central shaft, and a stopping device is arranged at first and second ends of the central shaft, on each side of the combined first and second body part, preventing the first body part and the second body part to slide in a longitudinal direction along the central shaft. The central shaft may be integrated in the first body part or in the second body part.

The first and/or the second body part may be arranged to rotate freely relative to each other, e.g., at least in a transition from the initial configuration to the attachment configuration.

The rotation of the first body part in the first actuating direction can cause, such as allow, the first attachment part to rotate relative to the second attachment part. For example, the first attachment part can translate in a first direction about a first axis in relation to the first body part.

The rotation of the second body part in the second actuating direction can cause, such as allow, the second attachment part to rotate relative to the first attachment part. For example, the second attachment part can translate in a first direction about a first axis in relation to the second body part.

For example, actuator mechanism can be configured to move the first attachment part in relation to the second attachment part, such as configured to move the first distal end towards and/or away from the second distal end. For example, the actuator mechanism is configured to translate the first attachment part in the first direction about the first axis and the second attachment part in the first direction about the first axis. The actuator mechanism, such as the resilient part, may be configured to rotate the first attachment part about the first axis in the first direction in relation to the first body part. The actuator mechanism, such as the resilient part, may be configured to rotate the second attachment part about the second axis in the first direction in relation to the second body part.

In one or more example delivery devices, the actuator mechanism is configured to move the first distal end towards the second distal end, such as from the initial configuration to the attachment configuration. To move the first distal end towards the second distal end may be understood as reducing a distance between the first distal end and the second distal end. To move the first distal end towards the second distal end may be understood as reducing an angle between the first attachment axis and the second attachment axis, such as reducing an angle between the first attachment part and the second attachment part. In one or more example drug delivery devices, the actuator mechanism reduces the distance between the first distal end and the second distal end during rotation of the first body part with respect to the second body part around the central axis. In certain example embodiments, the drug delivery device may be in the attachment configuration when the first distal end is closest to the second distal end.

Thus, the attachment parts may be adapted to come into contact and/or penetrate tissue of the gastrointestinal tract. The rotation of the body parts relative to each other using a resilient force may translate the attachment parts in such a way that they are capable of e.g., penetrating or pinching the mucous membrane in order to fix the drug delivery device at a location in the gastrointestinal tract, such as the stomach and/or gastrointestinal tissues and/or intestines. When the rotational force is applied to both the first and the second body part, the first attachment part may come into contact with the surface to be attached to, and where the rotational force applied to the second body part may cause the second attachment part to come into contact with the same surface, where the first attachment part provides a force, while the second attachment part provides a counter force to the first attachment part, so that the force is applied in such a manner that the first attachment part is forced in a direction towards the second attachment part, or vice versa. This can be known as the attachment configuration of the drug delivery device.

When the first attachment part and/or the second attachment part penetrates or pinches the biological tissue due to the rotation between the first body part and the second body part (the first distal end moving towards the second distal end, the respective penetration point(s) in the biological tissue may be utilized to deliver a drug substance from the drug delivery device into the biological tissue, and where the drug substance may be introduced into the biological tissue that is beyond the mucous membrane. Thereby, the drug substance may enter the bloodstream more easily than if the drug substance is released in the stomach or intestinal lumen, and the drug delivery may be more effective. An example of this is when the drug substance is insulin, where insulin may degrade inside the gastrointestinal tract and is not capable of being absorbed from the gastrointestinal tract, but where a mucous membrane has been penetrated, and the insulin released through the penetrated gastrointestinal wall, the insulin will remain intact and reach the bloodstream of the user via the blood vessels in the intestinal layer beyond the mucous membrane (surface).

Within the context of the present description the term “rotational force” may be seen as torque, moment, moment of force, rotational force or “turning effect”. Another definition of the term “rotational force” may be the product of the magnitude of the force and the perpendicular distance of the line of action of force from the axis of rotation. The rotational force may be seen as the force which is transferred from the resilient part to the attachment members of the drug delivery device via the body parts.

In one or more example drug delivery devices, when the first body part and/or the second body part rotate with respect to one another about the central axis, the first attachment part and/or the second attachment part can rotate out of their respective recesses (e.g., first recess and second recess) due to the rotation of the first body part and/or the second body part. For example, the first attachment part can translate in a first direction about the first axis. The second attachment part can translate in a second direction about the second axis.

The continued rotation of the first body part and/or the second body part then causes the first attachment part and/or the second attachment part to pierce tissue to hold the drug delivery device in place. Thus, in an initial configuration, the first attachment part may be located within the first recess. After rotation to the attachment configuration, the first attachment part may be located at least partially outside of the first recess. Alternatively, the first attachment part and/or the second attachment part may not be located in a recess, but may be located on an outer surface of the drug delivery device.

As an example, the actuator mechanism may be configured to rotate the first body part in a first actuating direction. The second body part may remain stationary, but in other examples may rotate as well. This rotation of the first body part may force the first attachment part to rotate in the first direction about the first axis in relation to the first body part. The first direction may be opposite of the first actuating direction, for example, the first body part is rotating in one direction and the first attachment part is rotated opposite that direction. At a certain point, the first attachment part stop rotating in the first direction about the first axis while the first body part continues to rotate in the first actuating direction. This continued rotation may bring the first distal end closer to the second distal end, such as to pierce tissue.

In one or more example drug delivery devices, the drug delivery device comprises a locking mechanism. The locking mechanism may be configured to prevent rotation of the first body part with respect to the second body part, e.g. in the initial configuration. The locking mechanism can be configured to release, such as dissolve, allowing rotation. The locking mechanism may be, for example, a cover and/or a collar around an outer surface of the drug delivery device. Other locking mechanisms can be used, such as a dissolvable plug, and the particular locking mechanism is not limiting.

In one or more example drug delivery devices, the drug delivery device may transition from an initial configuration to an attachment configuration, and from the attachment configuration to the release configuration. Advantageously, this can allow the drug delivery device to attach to a patient, deliver an active drug substance, and then release from the patient.

In the initial configuration, the first attachment part may be in the first initial position and/or the second attachment part may be in the second initial position. For example, the first initial position may be where the first attachment part, such as the first distal end, is located within the first recess. A similar position can be taken for the second attachment part. Further, the actuating mechanism may be configured to, but prevented from, rotating the first body part and/or the second body part with respect to one another. This may be the most compact configuration of the drug delivery device.

The drug delivery device can then transition from the initial configuration to the attachment configuration. This transition may occur due to actuation of the actuator mechanism. For example, due to a locking mechanism releasing. The transition from the initial configuration to the attachment configuration may include rotating the first body part in relation to the second body part in a first actuating direction about the central axis. In other words, the first body part and the second body part can rotate with respect to one another.

The transition from the initial configuration to the attachment configuration may also include translating the first attachment part in a first direction about a first axis in relation to the first body part. In other words, the first attachment part can translate out of the first recess. The transition from the initial configuration to the attachment configuration may also include translating the second attachment part in a first direction about a second axis in relation to the second body part. In other words, the second attachment part can translate out of the second recess. The attachment configuration can be configured for attaching the drug delivery device to a patient.

The first direction about the first axis may be opposite of the first actuating direction. For example, as the first body part is rotating in one direction, the first attachment part may rotate in the opposite direction.

The first direction about the second axis may be opposite of the second actuating direction. For example, as the second body part is rotating in one direction, the second attachment part may rotate in the opposite direction.

Further, the first distal end can translate towards the second distal end. While the first attachment part may be rotating away from the second attachment part, the rotation of the first body part with respect to the second body part may bring the first distal end towards the second distal end.

In one or more example drug delivery devices, translating the first attachment part in the first direction about the first axis can include rotating the first attachment part in the first direction about the first axis. Similarly, translating the second attachment part in the first direction about the second axis can include rotating the second attachment part in the first direction about the second axis. The first direction about the first axis may be the opposite as the first direction about the second axis, so that the first distal end moves closer to the second distal end.

The drug delivery device can be configured to translate from the attachment configuration to the release configuration. This may occur after a time period when the drug delivery device is in the attachment configuration. Discussed herein are a number of mechanisms for transitioning the drug delivery device from the attachment configuration to the release configuration.

In one or more example drug delivery devices, translating the first attachment part in the first direction about the first axis comprises rotating the first attachment part, e.g., at least 5 degrees, in the first direction about the first axis.

In one or more example drug delivery devices, the transition from the attachment configuration to the release configuration can include translating the first attachment part in the first direction about the first axis. The transition from the attachment configuration to the release configuration can include rotating the first attachment part, e.g., at least 5 degrees, in the first direction about the first axis.

For example, the first attachment part may stop at a first attachment position after translating in the first direction about the first direction when the drug delivery device is in the initial position. For example, the first attachment part may abut against a stop component, such as the first dissolvable component discussed below. The first attachment part may continue translating in the first direction about the first axis in the transition to the release configuration and stop in the first release position when the drug delivery device is in the release configuration. The translation of the first attachment part in the first direction about the first axis can bring the first distal end farther from the second distal end, e.g. after bringing the first distal end closer to the second distal end. This can loosen the drug delivery device and allow for detachment.

The first attachment part may continue translation in the first direction about the first action based on rotation of the first body part in the first actuating direction. The first attachment part may continue translation in the first direction about the first axis without any rotation of the first body part in the first actuating direction.

In one or more example drug delivery devices, the transition from the attachment configuration to the release configuration can include rotating the first body part in relation to the second body part in the first detachment direction, e.g., opposite the first actuating direction

For example, the rotation of the first body part in relation to the second body part in the first actuating direction can bring the first distal end closer to the second distal end. The rotation of the first body part in relation to the second body part in the first detachment direction can bring the first distal end farther form the second distal end. This can loosen the drug delivery device and allow for detachment.

In one or more example drug delivery devices, one or more components can be used to prevent one or more of the transitions discussed, such as the transition from the initial configuration the attachment configuration and/or the transition from the attachment configuration to the release configuration. In one or more example drug delivery devices, one or more dissolvable components can be used to prevent one or more of the transitions discussed, such as the transition from the initial configuration the attachment configuration and/or the transition from the attachment configuration to the release configuration.

In one or more example drug delivery devices, the drug delivery device can include at least one dissolvable component. The dissolvable component can be configured to prevent transitioning from the attachment configuration to the release configuration.

The first attachment part can include one or more of the at least one dissolvable component, e.g. a first dissolvable component. The first body part can include one or more of the at least one dissolvable component. The second attachment part can include one or more of the at least one dissolvable component, e.g. a second dissolvable component. The second body part can include one or more of the at least one dissolvable component.

The dissolvable component(s), such as the first dissolvable component, the second dissolvable component, the dissolvable material, and/or the actuator dissolvable component, can be one or more of dissolvable, biodegradable, degradable, and destructible. A dissolvable component, such as the first dissolvable component, the second dissolvable component, the dissolvable material, and/or the actuator dissolvable component, can be a component configured to erode. A dissolvable component, such as the first dissolvable component, the second dissolvable component, the dissolvable material, and/or the actuator dissolvable component, can be a component configured to lose one or more of strength, density, tension, and elasticity, e.g. upon contact with a fluid. A dissolvable component, such as the first dissolvable component, the second dissolvable component, the dissolvable material, and/or the actuator dissolvable component, can be a swellable component. The dissolvable component(s), such as the first dissolvable component, the second dissolvable component, the dissolvable material, and/or the actuator dissolvable component may be a component capable of weakening. The dissolvable component(s), such as the first dissolvable component, the second dissolvable component, the dissolvable material, and/or the actuator dissolvable component may only weaken and not fully dissolve. The dissolvable component(s), such as the first dissolvable component, the second dissolvable component, the dissolvable material, and/or the actuator dissolvable component may weaken upon contact with a fluid.

The dissolvable component(s), such as the first dissolvable component, the second dissolvable component, the dissolvable material, and/or the actuator dissolvable component, can be fully dissolvable. The dissolvable component(s), such as the first dissolvable component, the second dissolvable component, the dissolvable material, and/or the actuator dissolvable component, can be partially dissolvable. The dissolvable component(s), such as the first dissolvable component, the second dissolvable component, the dissolvable material, and/or the actuator dissolvable component, can include one or more dissolvable portions.

The dissolvable component(s), such as the first dissolvable component, the second dissolvable component, the dissolvable material, and/or the actuator dissolvable component, can be considered a plug. The dissolvable component(s), such as the first dissolvable component, the second dissolvable component, the dissolvable material, and/or the actuator dissolvable component, can be considered a stop. The dissolvable component(s), such as the first dissolvable component, the second dissolvable component, the dissolvable material, and/or the actuator dissolvable component, can be considered a backstop.

In one or more example drug delivery devices, the at least one dissolvable component can include a first dissolvable component. The first dissolvable component can be configured to prevent the first attachment part from transitioning from a first attachment position in the attachment configuration to a first release position in the release configuration.

For example, the first dissolvable component can be configured to act as a backstop, stopping the first attachment part in the first attachment position.

The first attachment part can start in the first initial position. Upon actuation of the actuator, the first attachment part can transition to the first attachment position. Upon dissolution of the first dissolvable component, the first attachment part can transition to the first release position.

In one or more example drug delivery devices, the first dissolvable component, in the attachment configuration of the drug delivery device, can prevent rotation of the first attachment part in the first direction.

For example, the first attachment part may be stopped from moving, such as translating and/or rotating, in the first direction (such as further in the first direction) about the first axis by the first dissolvable component. The first attachment part may be in the first attachment position when the first attachment part abuts, such as is in contact with, the first dissolvable component. The first dissolvable component can act as a barrier to prevent the first attachment part from translating in the first direction about the first axis from the first initial position. The first dissolvable component can be arranged in and/or form a part of the first attachment part.

In one or more example drug delivery devices, the at least one dissolvable component can include a second dissolvable component. The second dissolvable component can be configured to prevent the second attachment part from transitioning from a second attachment position in the attachment configuration to a second release position in the release configuration.

For example, the second attachment part may be stopped from moving, such as translating and/or rotating, further in the first direction about the second axis by the second dissolvable component. The second attachment part may be in the second attachment position when the second attachment part abuts, such as is in contact with, the second dissolvable component. The second dissolvable component can act as a barrier to prevent the second attachment part from translating in the first direction about the second axis from the second initial position. The second dissolvable component can be arranged in and/or form a part of the second attachment part.

The second attachment part can start in the second initial position. Upon actuation of the actuator, the second attachment part can transition to the second attachment position. Upon dissolution of the second dissolvable component, the second attachment part can transition to the second release position.

The second dissolvable component can include any and/or all features of the first dissolvable component. The second dissolvable component can be configured to be dissolvable at the same rate as the first dissolvable component. The second dissolvable component can be configured to dissolve at a different rate than the first dissolvable component. The second dissolvable component can be in connection with the first dissolvable component. The second dissolvable component can be separate from the first dissolvable component.

In one or more example drug delivery devices, the at least one dissolvable component includes an actuator dissolvable component. The actuator mechanism, in the attachment configuration, can be connected to one or both of the first body part and the second body part via the actuator dissolvable component. In the release configuration, the actuator mechanism can be disengaged from one or both of the first body part and the second body part.

For example, the actuator dissolvable component can be a protrusion, such as an extension. The actuator dissolvable component may extend radially inwards from an inner surface of the first body part and/or the second body part. The actuator mechanism may be connected to, such as hooked on, surrounded, etc., the actuator dissolvable component. Prior to dissolving of the actuator dissolvable component, the actuator mechanism may still be applying a rotation force to the first body part and/or the second body part.

Upon dissolution, the actuator mechanism can be disconnected, such as disengaged, such as released, from the actuator dissolvable component. Accordingly, the actuator mechanism can be disengaged from the first body part and/or the second body part. The actuator mechanism may no longer be applying a rotational force on the first body part and/or the second body part, thereby allowing the first body part to rotate in relation to the second body part in a first disenchantment direction opposite the first actuating direction.

In one or more example drug delivery devices, the actuator dissolvable component can be located on a radially inward facing surface of the first body part and/or the second body part.

In one or more example drug delivery devices, the actuator dissolvable component can be located on a radially inward facing surface of the first body part. In one or more example drug delivery devices, the actuator dissolvable component can be located on a radially inward facing surface of the second body part.

In one or more example drug delivery devices, the actuator dissolvable component can be located on a central shaft extending through the drug delivery device along the central axis.

The central shaft may be part of the first body part and/or the second body part. The central shaft may be separate from the first body part and/or the second body part.

For example, the actuator dissolvable component can be a protrusion, such as an extension. The actuator dissolvable component may extend radially outwards from the central shaft. The actuator mechanism may be connected to, such as hooked on, surrounded, etc., the actuator dissolvable component. Prior to dissolving of the actuator dissolvable component, the actuator mechanism may still be applying a rotation force to the first body part and/or the second body part.

The actuator dissolvable component may be an inner actuator dissolvable component, such as a protrusion extending radially inwards from the first body part and/or the second body part. The actuator dissolvable component may be an outer actuator dissolvable component, such as a protrusion extending radially outwards from the central shaft. The actuator dissolvable component may be both an inner actuator dissolvable component and an outer actuator dissolvable component.

In one or more example drug delivery devices, the actuator mechanism can include a dissolvable material. The actuator mechanism can be configured to prevent the rotating the first body part in relation to the second body part in the first detachment direction in the attachment configuration.

The actuator mechanism may be the at least one dissolvable component. The actuator mechanism may include a dissolvable material. Prior to dissolution, the actuator mechanism may provide a rotational force on the first body part and/or the second body part in the actuating direction. Upon dissolving of the dissolvable material, the actuator mechanism may not provide the rotational force on the first body part and/or the second body part in the actuating direction, which can allow the first body part to rotate in the first detachment direction.

The actuator mechanism may be fully made of the dissolvable material. The actuator mechanism may be partially made of the dissolvable material. The actuator mechanism can include one or more sections of the dissolvable materials.

In one or more example drug delivery devices, a dissolution of the actuator mechanism or the actuator dissolvable component can reduce a force applied by the actuator mechanism between the first body part and the second body part. In other words, an attachment force, AF, applied by the actuator mechanism between the first body part and the second body part in the attachment configuration may be larger than a release force, RF, applied by the actuator mechanism between the first body part and the second body part in the release configuration. In one or more example drug delivery devices, RF<0.5AF, e.g. RF<0.1AF.

The dissolution of the actuator mechanism or the actuator dissolvable component can eliminate the force applied by the actuator mechanism between the first body part and the second body part. This can allow the first body part to rotation in the first detachment direction.

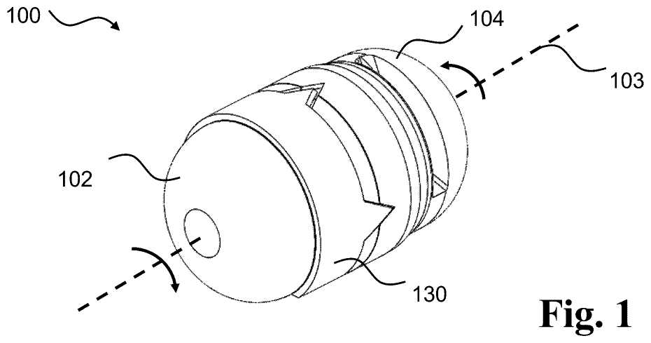

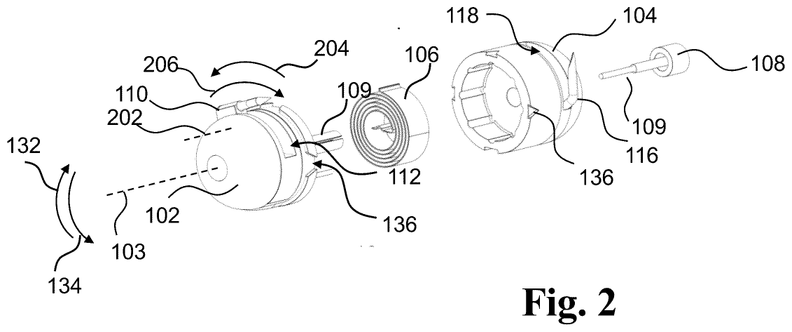

FIGS. 1 and 2 show views of a drug delivery device in accordance with the disclosure. In particular, the drug delivery device 100 can include a first body part 102, a second body part 104, a first attachment part 110 connected to the first body part 102 and having a first distal end and a second attachment part 116 connected to the second body part 104 and having a second distal and an actuator mechanism 106 configured to rotate one of the first body part 102 and/or the second body part 104 about a central axis 103. The drug delivery device 100 is configured to transition from an initial configuration as illustrated in FIG. 1 to an attachment configuration, and from the attachment configuration to a release configuration. Transition from the initial configuration to the attachment configuration includes translating the first attachment part 110 in a first direction 204 about a first axis 202 in relation to the first body part 102, and rotating the first body part 102 in relation to the second body part 104 in a first actuating direction 132 about the central axis 103. The transition from the attachment configuration to the release configuration can include one or more of translating the first attachment part 110 in the first direction 204, and rotating the first body part 102 in relation to the second body part 104 in a first detachment direction 136 opposite the first actuating direction 134. As shown, the drug delivery device 100 can include a first body part 102, a second body part 104, and a central axis 103 extending through the first body part 102 and the second body part 104. The first body part 102 and the second body part 104 can rotate with respect to one another around the central axis 103. The drug delivery device 100 can further include a locking mechanism 130. FIG. 1 illustrates the drug delivery device in an initial configuration. The first body part 102 can be configured to rotate in relation to the second body part 104 in a first actuating direction 134 about the central axis 103. Optionally, the first body part 102 can be configured to rotate in relation to the second body part 104 in a first detachment direction 134 about the central axis 103.

FIG. 2 shows an exploded view of the drug delivery device 100. As shown, the first body part 102 includes a first recess 112 while the second body part 104 includes a second recess 118. A first attachment part 110 can be located within the first recess 112 and a second attachment part 116 can be located within the second recess 118. FIG. 1 illustrates the first attachment part 110 in the first initial position and the second attachment part 116 in the second initial position.

For example, the first attachment part 110 can be translatable, such as rotatable, about a first axis 202. For example, the first attachment part 110 can be translatable, such as rotatable, in a first direction 204 about the first axis 202 in relation to the first body part 102. Optionally, the first attachment part 110 can be translatable, such as rotatable, in a second direction 206 about the first axis 202 in relation to the first body part 102. Translating the first attachment part 110 in the first direction 132 about the first axis 202 can include rotating the first attachment part 110 in the first direction 132 about the first axis 202.

FIG. 2 further show an axle 108 for rotation of the first body part 102 and the second body part 104, such as through the use of an actuator mechanism 106 for rotating the first body part 102 or the second body part 104 with respect to one another about the central axis 103. Further, FIG. 2 shows a central shaft 109 extending through the drug delivery device 110 along the central axis 103. A locking mechanism 130 can prevent such rotation until the locking mechanism 130 is removed, such as by dissolving or biodegradation.

The first body part 102 can be configured to rotate in relation to the second body part 104 in a first actuating direction 134 about the central axis 103. Optionally, the first body part 102 can be configured to rotate in relation to the second body part 104 in a first detachment direction 134 about the central axis 103.

The drug delivery device 100 can include at least one dissolvable component configured to prevent transitioning from the attachment configuration to the release configuration.

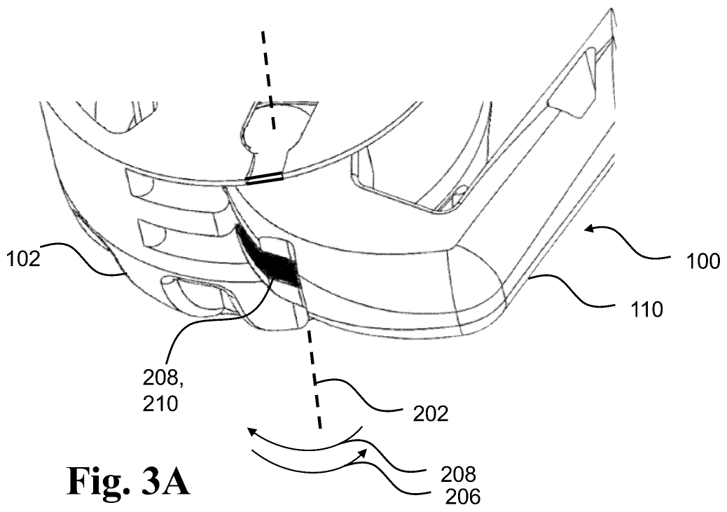

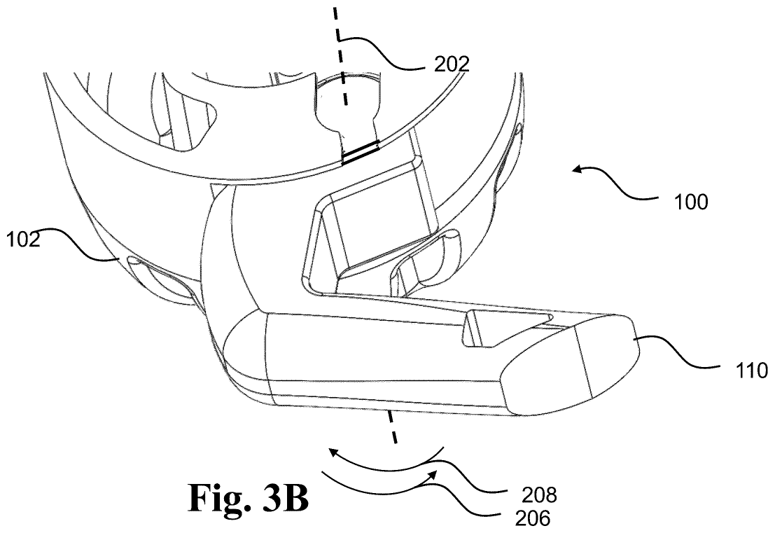

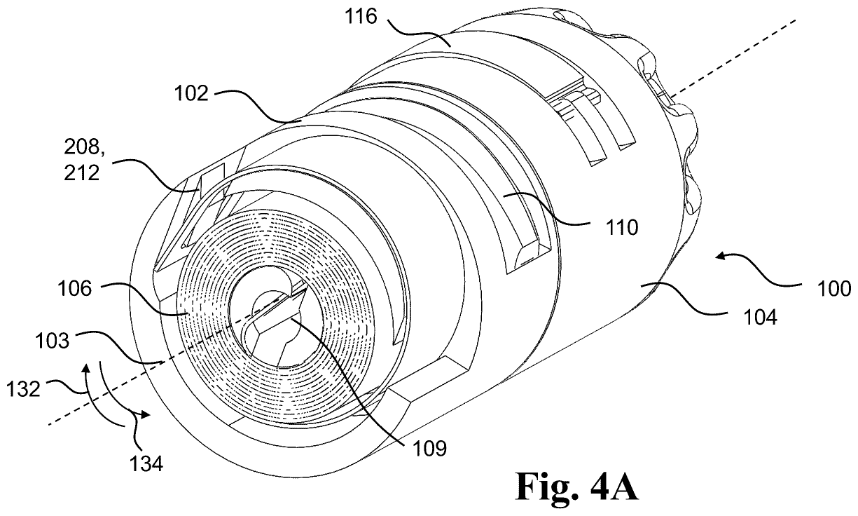

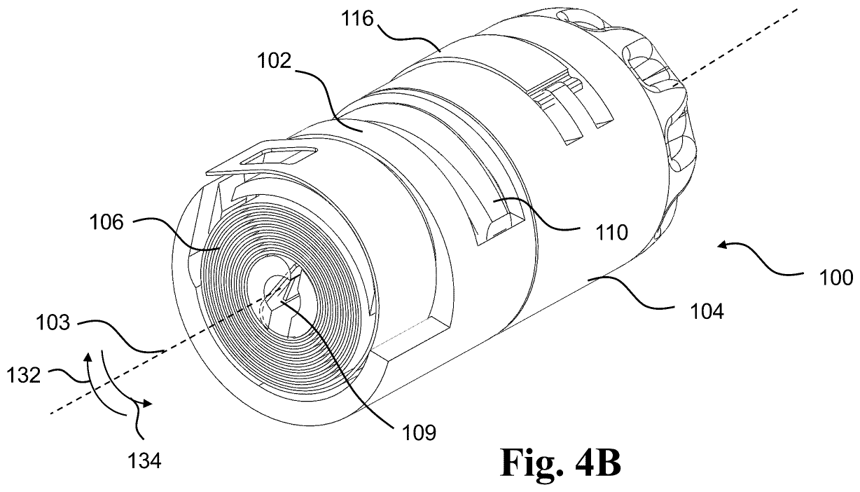

Variations of the at least one dissolvable component are discussed with respect to FIGS. 3A-4B.

FIGS. 3A-3B illustrate a drug delivery device including at least one dissolvable component 206. The drug delivery device shown in FIGS. 3A-3B can include any and/or all of the components of drug delivery device 100, and will hereby use the same reference number.

FIG. 3A illustrates the drug delivery device 100 in the attachment configuration. Accordingly, the first attachment part 110 has translated in a first direction 204 about a first axis 202 in relation to the first body part 102, and the first body part 102 has rotated in relation to the second body part in a first actuating direction 132 about the central axis 103.

As shown, the first attachment part is in the first attachment position. The first attachment part 110 abuts against at least one dissolvable component 208, such as the first dissolvable component 210. The first dissolvable component 210 prevents the first attachment part 110 from continuing to translate in the first direction 208. For example, the first dissolvable component 210 prevents the drug delivery device 100 from transitioning from the attachment configuration to a release configuration. Specifically, the first dissolvable component 210 is configured to prevent the first attachment part 110 from transitioning from a first attachment position in the attachment configuration to a first release position in the release configuration. For example, the first dissolvable component 210, in the attachment configuration of the drug delivery device 100, prevents rotation of the first attachment part 110 in the first direction.

FIG. 3B illustrates the drug delivery device 100 after the first dissolvable component 210 has dissolved. In particular, the transition from the attachment configuration to the release configuration includes translating the first attachment part 110 in the first direction 204 about the first axis 202.

As shown, the first attachment part 110 can continue to translate in the first direction 208 about the first body part 102 into the first release position. In particular, FIG. 3B illustrates a release configuration of the drug delivery device 100. The first attachment part 110 has continued to move away from the second attachment part 116, shown in FIG. 2, thereby loosening the drug delivery device 110 and allowing the drug delivery device 100 to release from tissue.

The second attachment part 116 can include and/and or all of the features discussed above with respect to the first attachment part 110 discussed above with respect to FIGS. 3A-3B.

For example, the at least one dissolvable component 208 can include a second dissolvable component. The second dissolvable component can be configured to prevent the second attachment part 116 from transitioning from a second attachment position in the attachment configuration to a second release position in the release configuration.

FIGS. 4A-4B illustrate a drug delivery device including at least one dissolvable component 206. The drug delivery device shown in FIGS. 4A-4B can include any and/or all of the components of drug delivery device 100, and will hereby use the same reference number. FIGS. 4A-4B do not illustrate movement of the relative attachment parts 110, 116 for convenience sake.

FIG. 4A illustrates the drug delivery device 100 in the attachment configuration. Accordingly, the first attachment part 110 has translated in a first direction 204 about a first axis 202 in relation to the first body part 102, and the first body part 102 has rotated in relation to the second body part in a first actuating direction 132 about the central axis 103.

As shown, the actuator mechanism 106 is connected to the first body part 102 via at least one dissolvable component 208, such as actuator dissolvable component 212. The actuator mechanism 106, in the attachment configuration, can be connected to one or both of the first body part 102 and the second body part 104 via the actuator dissolvable component 212, and wherein, in the release configuration, the actuator mechanism 106 is disengaged from one or both of the first body part 102 and the second body part 104.

The actuator dissolvable component 212 allows the actuator mechanism 106 to continue applying a rotational force to rotate the first body part 102 in the first actuating direction 132 about the central axis 103. For example, the actuator dissolvable component 212 prevents the drug delivery device 100 from transitioning from the attachment configuration to a release configuration.

FIG. 4B illustrates the drug delivery device 100 after the actuator dissolvable component 212 has dissolved. In particular, the transition from the attachment configuration to the release configuration includes rotating the first body part 102 in relation to the second body part 104 in the first detachment direction opposite 134 the first actuating direction 132.

Accordingly, the actuator mechanism 106 is disengaged from the first body part 102. The actuator mechanism 106 no longer applies a rotational force to rotate the first body part 102 in the first actuating direction 132. Accordingly, the first body part 102 can rotate in relation to the second body part 104 in a first detachment direction 134 opposite the first actuating direction 132.

As shown, the actuator dissolvable component 106 can be located on a radially inward facing surface of the first body part 102. The actuator dissolvable component 106 can be located on a radially inward facing surface of the second body part 104. Optionally, or instead, the actuator dissolvable component 212 can be located on a central shaft 109 extending through the drug delivery device 100 along the central axis 103.

Optionally, the actuator mechanism 106 can include a dissolvable material. The actuator mechanism 106 can be configured to prevent the rotating the first body part in relation to the second body part in the first detachment direction in the attachment configuration. The drug delivery device 100 upon dissolution of the dissolvable material may act in a similar manner as discussed with respect to FIGS. 4A-4B.

For example, a dissolution of the actuator mechanism or the actuator dissolvable component reduces a force applied by the actuator mechanism between the first body part and the second body part.

Also disclosed are delivery devices, methods, and compositions according to any of the following items.

Item 1. A drug delivery device comprising:
a first body part;
a second body part;
a first attachment part connected to the first body part and having a first distal end and a second attachment part connected to the second body part and having a second distal end; and
an actuator mechanism configured to rotate one of the first body part and/or the second body part about a central axis;
wherein the drug delivery device is configured to transition from an initial configuration to an attachment configuration, and from the attachment configuration to a release configuration;
wherein the transition from the initial configuration to the attachment configuration comprises translating the first attachment part in a first direction about a first axis in relation to the first body part, and rotating the first body part in relation to the second body part in a first actuating direction about the central axis; and
wherein the transition from the attachment configuration to the release configuration comprises one or more of translating the first attachment part in the first direction, and rotating the first body part in relation to the second body part in a first detachment direction opposite the first actuating direction.
   Item 2. Drug delivery device according to Item 1, wherein translating the first attachment part in the first direction about the first axis comprises rotating the first attachment part in the first direction about the first axis.
   Item 3. Drug delivery device according to any one of the preceding Items, wherein the transition from the attachment configuration to the release configuration comprises translating the first attachment part in the first direction about the first axis.
   Item 4. Drug delivery device according to any one of the preceding Items, wherein the transition from the attachment configuration to the release configuration comprises rotating the first body part in relation to the second body part in the first detachment direction opposite the first actuating direction.
   Item 5. Drug delivery device according to any one of the preceding Items, wherein the drug delivery device comprises at least one dissolvable component configured to prevent transitioning from the attachment configuration to the release configuration.
   Item 6. Drug delivery device according to Item 5, wherein the at least one dissolvable component comprises a first dissolvable component, wherein the first dissolvable component is configured to prevent the first attachment part from transitioning from a first attachment position in the attachment configuration to a first release position in the release configuration.
   Item 7. Drug delivery device according to Item 6, wherein the first dissolvable component, in the attachment configuration of the drug delivery device, prevents rotation of the first attachment part in the first direction.
   Item 8. Drug delivery device according to any one of Items 5-7, wherein the at least one dissolvable component comprises a second dissolvable component, wherein the second dissolvable component is configured to prevent the second attachment part from transitioning from a second attachment position in the attachment configuration to a second release position in the release configuration.
   Item 9. Drug delivery device according to any one of Items 5-8, wherein the at least one dissolvable component comprises an actuator dissolvable component, wherein the actuator mechanism, in the attachment configuration, is connected to one or both of the first body part and the second body part via the actuator dissolvable component, and wherein, in the release configuration, the actuator mechanism is disengaged from one or both of the first body part and the second body part.
   Item 10. Drug delivery device according to Item 9, wherein the actuator dissolvable component is located on a radially inward facing surface of the first body part and/or the second body part.
   Item 11. Drug delivery device according to Item 9, wherein the actuator dissolvable component is located on a central shaft extending through the drug delivery device along the central axis.
   Item 12. Drug delivery device according to any one of Items 1-11, wherein the actuator mechanism comprises a dissolvable material, and wherein the actuator mechanism is configured to prevent the rotating the first body part in relation to the second body part in the first detachment direction in the attachment configuration.
   Item 13. Drug delivery device according to any one of Items 9-12, wherein a dissolution of the actuator mechanism or the actuator dissolvable component reduces a force applied by the actuator mechanism between the first body part and the second body part.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100 drug delivery device
102 first body part
103 central axis
104 second body part
106 actuator mechanism
108 axle
109 central shaft
110 first attachment part
112 first recess
116 second attachment part
118 second recess
130 locking mechanism
132 first actuating direction
134 first detachment direction
202 first axis
204 first direction
206 second direction
208 at least one dissolvable component
210 first dissolvable component
212 actuator dissolvable component

1. A drug delivery device comprising:
a first body part;
a second body part;
a first attachment part connected to the first body part and having a first distal end and a second attachment part connected to the second body part and having a second distal end; and
an actuator mechanism configured to rotate one of the first body part and/or the second body part about a central axis;
wherein the drug delivery device is configured to transition from an initial configuration to an attachment configuration, and from the attachment configuration to a release configuration;
wherein the transition from the initial configuration to the attachment configuration comprises translating the first attachment part in a first direction about a first axis in relation to the first body part, and rotating the first body part in relation to the second body part in a first actuating direction about the central axis; and
wherein the transition from the attachment configuration to the release configuration comprises one or more of translating the first attachment part in the first direction, and rotating the first body part in relation to the second body part in a first detachment direction opposite the first actuating direction.

2-13. (canceled)
